(19) United States
(12) Patent Application Publication
(10) Pub. No.: US 2021/0128132 A1
(43) Pub. Date: May 6, 2021

(54) Orthopedic Retractor For Hip Replacement

(71) Applicant: Mitchell Klement, Hartland, WI (US)

(72) Inventor: Mitchell Klement, Hartland, WI (US)

(21) Appl. No.: 17/091,023

(22) Filed: Nov. 6, 2020

Related U.S. Application Data
(60) Provisional application No. 62/931,559, filed on Nov. 6, 2019.

(51) Int. Cl. A61B 17/02; A61F 2/46

(52) U.S. Cl. CPC A61B 17/025; A61B 2017/0046; A61B 2017/0275; A61F 2/4607

(57) ABSTRACT

An orthopedic retractor includes a handle having a first end and a second end, an acetabular component removably coupled to the second end of the handle, and a hook element having a first end removably coupled to the handle at a location between the first and second ends. The hook element also includes a second end and a curve disposed between the first and second ends. Further, the acetabular component is configured to interfit with an acetabulum for anchor and provide a fulcrum for the retractor.

### CROSS-REFERENCE TO RELATED APPLICATION

This application claims the benefit of U.S. Provisional Application No. 62/931,559, filed on Nov. 6, 2019, which is incorporated by reference herein in its entirety.

### BACKGROUND OF THE INVENTION

### 1. Field of the Invention

The present invention is related to an orthopedic retractor for use in a hip replacement procedure and, more particularly, to an orthopedic retractor that uses the acetabulum as a fulcrum.

### 2. Discussion of Related Art

End stage hip arthritis can be a devastating condition for patients that affect their quality of life and ability to perform activities of daily living. Hip replacement surgery is an excellent option for these patients with reproducible and successful outcomes. Many surgical approaches have been described to access the hip joint and perform the operation. One approach gaining popularity is the direct anterior approach.

The direct anterior approach is a muscle sparing approach that may allow faster recovery and better patient outcomes. Traditionally, the direct anterior hip procedure requires specialized equipment including procedure-specific retractors, hip implants, x-ray machines, expensive operating tables, and multiple assistants. Performing the anterior approach without special tables (e.g., on a regular operating table) and without x-ray may allow the procedure to be performed in multiple operating rooms, multiple hospitals, and by multiple surgeons with enhanced efficiency and less cost. Currently, to perform the anterior approach on a regular operating table multiple assistants or specialized retractor assemblies are required.

As such, there is a need in the art for a simple orthopedic retractor to allow surgeons to perform the direct anterior hip replacement surgery with less assistants and less specialized equipment.

### SUMMARY OF THE INVENTION

The present invention is related to an orthopedic retractor for use in a hip replacement procedure, such as, but not limited to, an anterior hip replacement procedure. The purpose of the current invention is to allow surgeons to perform the direct anterior hip replacement surgery with a simple retractor system and potentially less assistants. This can be used with or without the specialized operating table and assist the surgeon in particular with exposure, preparation of the femur, and minimizing soft tissue damage. Previous retractors for femoral exposure are positioned around the proximal femur and either use the surrounding skin/soft tissue for leverage or are anchored to apparatuses anchored to the table. Table based apparatuses use hooks on tension or hydraulic lifts to elevate the femur. The femoral retractor proposed herein would be the first retractor used in hip replacement surgery that is placed into the acetabulum for mechanical advantage and anchoring. This could be the native acetabulum if the surgeon prefers to prepare the femur first or the acetabular component if this has already been implanted. By anchoring the rector in the acetabulum this gives the assistant maximal leverage minimizing the work on the assistant and avoiding increased pressure on the skin and soft tissue. This also avoids the need for a table mount, potentially avoiding contamination and bulky apparatuses. While the direct anterior approach is highlighted herein, this retractor can be used with any hip approach and any operating table, such as, but not limited to any anterior-based hip approach, direct and other lateral-based hip approaches, Watson-Jones hip approach, Rottinger hip approach, anterior based muscle sparing, and posterior hip approaches.

According to an aspect of the invention, an orthopedic retractor includes a handle having a first end and a second end, an acetabular component removably coupled to the second end of the handle, and a hook element having a first end removably coupled to the handle at a location between the first and second ends. The hook element also includes a second end and a curve disposed between the first and second ends.

In accordance with another aspect of the invention, the handle includes a bend disposed between the first and second ends. The hook element is removably coupled to the handle at the bend of the handle. In addition, the handle includes a connection port disposed on an outer radius of the bend of the handle. The hook element includes an interfit element disposed at the first end of the hook element and configured to be disposed within the connection port to removably couple the hook element to the handle. Further, the connection port may be oriented tangential to the outer radius of the bend. Meanwhile, the interfit element extends from the first end of the hook element at an angle.

According to another embodiment of the invention, a method of using an orthopedic retractor includes coupling an acetabular component to a first end of a handle, disposing the acetabular component within a acetabulum, placing a hook element around a femur so that the femur is partially surrounded by a curve of the hook element, and coupling a first end of the hook element to the handle. In turn, the handle is used to pivot the retractor about the acetabulum to move the femur.

In accordance with another aspect of the invention, the first end of the hook element is coupled to an outer radius of a bend of the handle disposed between the first and second ends of the handle.

### DESCRIPTION OF THE DRAWINGS

The drawings illustrate preferred embodiments presently contemplated for carrying out the invention.

In the drawings:

FIG. 1 is a side view of a retractor, according to an embodiment of the invention;

FIG. 2 is an exploded perspective view of the retractor of FIG. 1;

FIG. 3 is a perspective view of the retractor of FIG. 1 engaged with an acetabulum and femur;

FIG. 4 is a side view of a retractor, according to another embodiment of the invention; and

FIG. 5 is an exploded perspective view of the retractor of FIG. 4.

### DETAILED DESCRIPTION OF THE PREFERRED EMBODIMENTS

The present invention and the various features and advantageous details thereof are explained more fully with reference to the non-limiting embodiment described in detail in the following description.

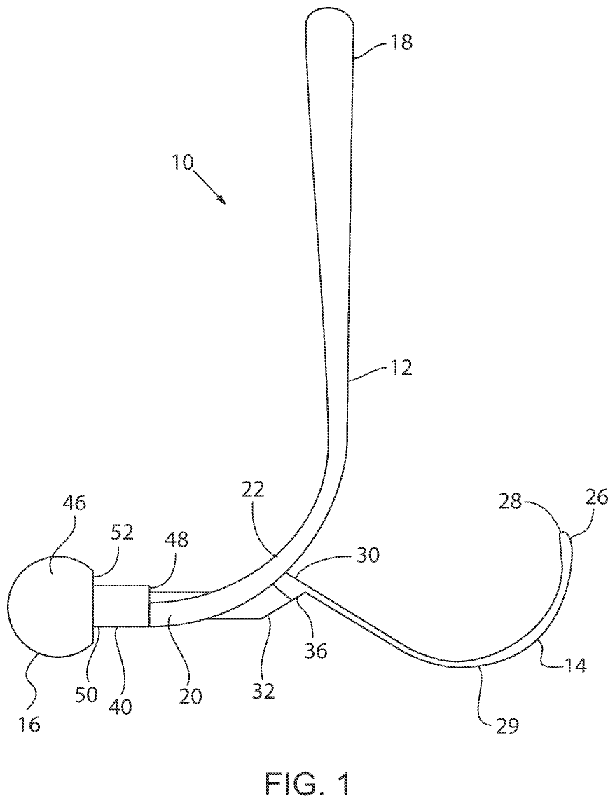

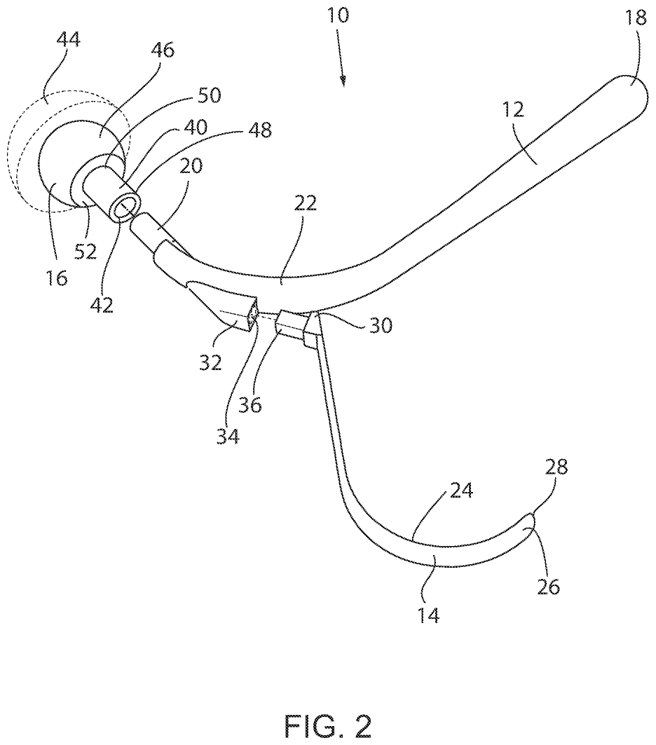

Referring to FIGS. 1 and 2, an orthopedic retractor 10 according to a representative embodiment of the invention. The retractor 10 includes a handle 12, a hook 14, and an acetabular component 16. In the representative embodiment of the invention, the handle 12 and includes a first end 18 and a second end 20 oriented perpendicular to the first end 18. In varying embodiments of the invention, the first end 18 of the handle 12 may be oriented at any angle with respect to the second end 20 of the handle 18. The handle 12 provides an ergonomic grip at the first end 18 for the assistant to hold the retractor 10 during surgery.

The handle 12 includes a bend 22 disposed between the first end 18 and the second end 20 in order to accommodate the change in orientation between the first and second end 18, 20. In the representative embodiment of the invention, the bend 22 is disposed adjacent the second end 20 of the handle 12. However, in varying embodiments of the invention, the bend 22 may be disposed at any location between the first and second ends 18, 20 of the handle 12.

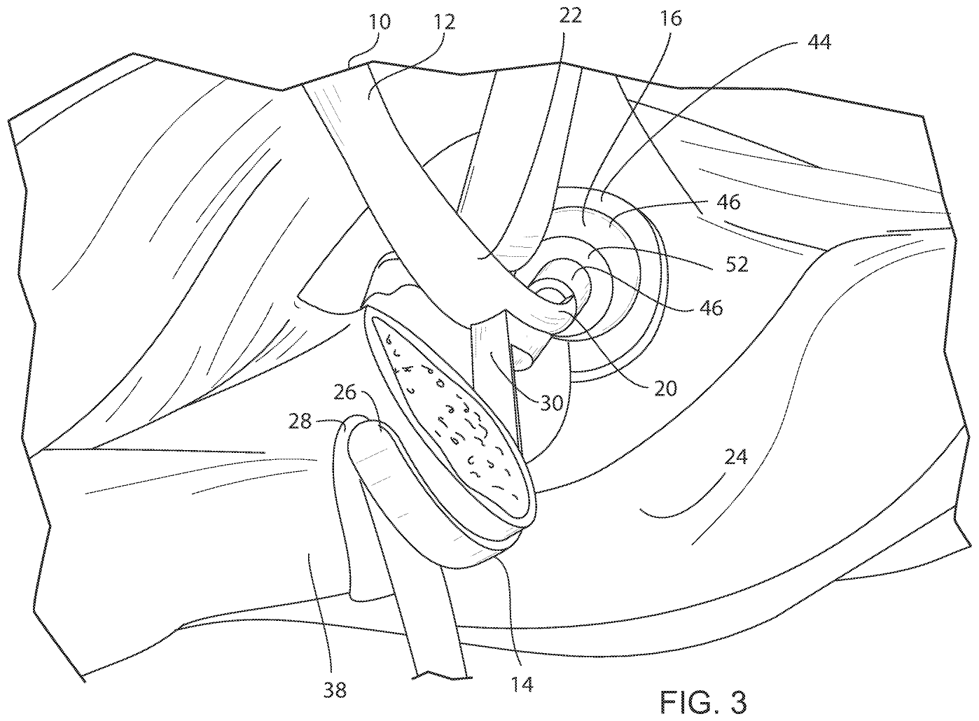

The hook 14 extends from the handle 12 and includes a gentle curve 24 and a blunt tip 26 at its first end 28. As shown in FIG. 2, a second end 30 of the hook 14 is detachably connected to the handle 12 at a location adjacent the bend 22. In the representative embodiment of the invention, the handle 12 includes a connection port 32 disposed on the outer radius of the bend 22. The connection port 32 is oriented tangential to the outer radius of the bend 22. The connection port 32 includes an opening 34 formed therein and configured to receive an interfit element 36 of the hook 14. The interfit element 36 extends at an angle from the second end 30 of the hook 14. In turn, when the interfit element 36 is disposed within the opening 34 of the connection port 32, the hook 14 extends outward from the outer radius of the bend 22 before gently curving at the curve 24. The curve 24 is configured to allow the hook 14 to be placed around a femur 38, which will be shown in FIG. 3. It is contemplated that the hook element 14 may come in a variety of orientations and sizes with varying angles at which it extends from the outer radius of the bend, varying radii of curvature of the curve 24 of the hook 14, and varying lengths and thicknesses.

The acetabular component 16 is detachable with the handle 12 by way of a connection element 40, such as a snap fit connection. In the representative embodiment of the invention, the connection element 40 includes an opening 42 formed in a first end 48 of the connection element 40 and configured to receive the second end 20 of the handle 12. Other embodiments of the invention may use other connection elements 40 to removably attach the acetabular component 16 to the handle 12.

The acetabular component 16 is configured to sit within the acetabulum 44 during use of the retractor 10. This will be further described with respect to FIG. 3. The acetabular component 16 may be in the form of a ball 46 that comes in various sizes to accommodate different sized acetabulum and acetabular components. For instance, sizes of the ball 46 may include 32 mm, 36 mm, 40 mm, 44 mm, 46 mm, 50 mm, and other commonly used sizes in hip replacement surgery. In turn, the connection element 40 of the acetabular component 16 may extend outward from the ball 46 of the acetabular component 16. As shown in FIG. 2, a second end 50 of the connection element 40 may be attached to the ball 46. In the representative embodiment of the invention, the second end 50 of the connection is coupled to a flat surface 52 of the ball 46. In other embodiments of the invention, the ball 46 may be completely circular, with the second end 50 of the connection element 40 attached to a rounded outer surface of the ball 46.

It is contemplated that the handle 12 and hook 14 may be molded from a material such as, but not limited to, stainless steel. Further yet, the acetabular component 16 may be made from a material such as, but not limited to, industrial plastic.

Now referring to FIG. 3, the regulator 10 is shown in use. Once the artificial acetabulum 44 has been placed by the doctor, the acetabular component 16 is configured to be connected to the handle 12 and then placed within the acetabulum 44. Meanwhile, the hook 14 is configured to be placed around the proximal femur 38 so that the curve 24 of the hook 14 partially surrounds the femur 38. The hook 14 is then anchored to the handle 12 through the interfit connection 32. Once the retractor 10 has been put in place and the handle 12, hook 14, and acetabular component 16 are coupled to each other, the retractor 10 is able to pivot about the acetabulum 44 and use it as a fulcrum to move the femur 38 as needed. That is, a single assistant is able to use the handle 12 to pivot the retractor about the acetabular component 16 and the acetabulum and move the hook 14, which lifts the femur 38 as necessary. In turn, a single assistant is able to manipulate the femur 38 as needed to by the doctor performing the hip replacement. If the surgeon also prefers to prepare the femur prior to the acetabulum or the surgeon is performing a hemiarthroplasty (femur only) then the retractor can be similarly placed in the native bony acetabulum with the proper sized ball 16.

Upon completion of the hip replacement, the retractor 10 is able to be removed and disassembled by detaching the handle 12, hook 14, and acetabular component 16 from each other. The separate elements 12, 14, 16 of the retractor 10 may then be sanitized together or separately after completion of the hip replacement.

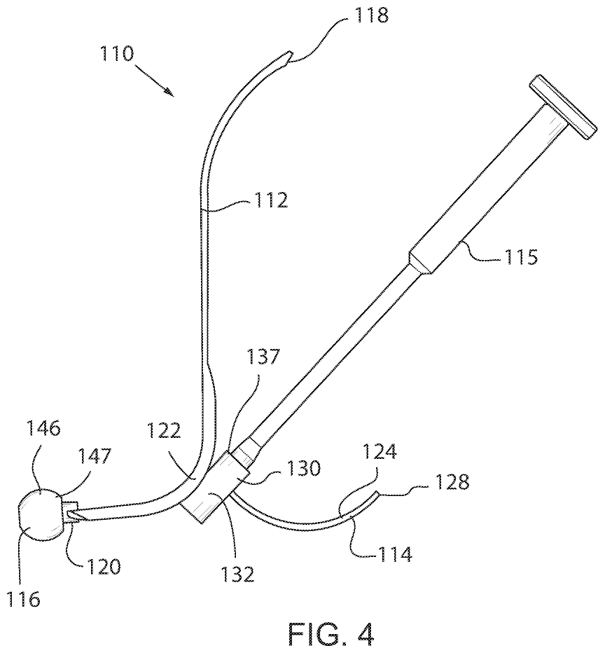

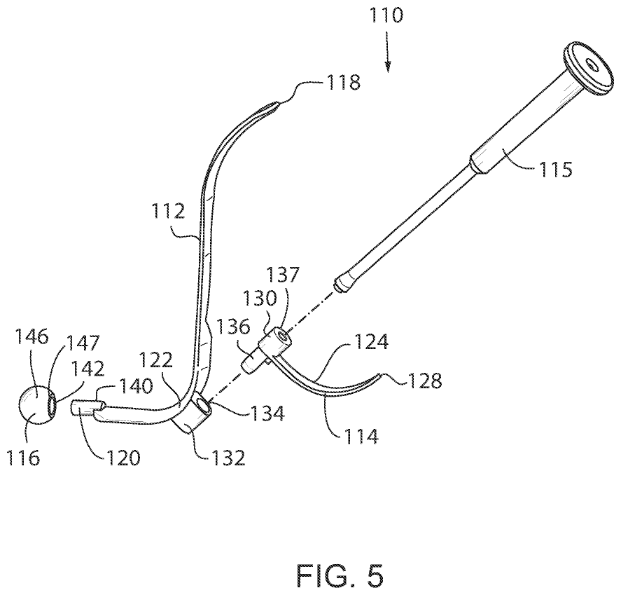

Referring now to FIGS. 4 and 5, an orthopedic retractor 110 according to another embodiment of the invention is shown. Similar to retractor 10, retractor 110 includes a handle 112, a hook 114, and an acetabular component 116. The handle 112 includes a first end 118 and a second end 120. As shown in FIGS. 4 and 5, the first end 118 of the handle 112 is oriented at an angle to the second end 120 of the handle 112. Preferably, the first end 118 of the handle 112 is oriented generally perpendicular to the second end 120 of the handle 112. Even more preferably, the first end 118 of the handle 112 is oriented perpendicular to the second end 120 of the handle 112. In turn, the first end 118 of the handle 112 provides a grip for the assistant to hold the retractor 110 during surgery.

In the representative embodiment of the invention, the handle 112 includes a bend 122 between the first end 118 of the handle 112 and the second end 120 of the handle 112. The bend 122 provides the change in direction of the handle 112 that results in the orientation of the first end 118 of the handle 112 being angled from the second end 120 of the handle 112.

The hook 114 of the retractor 110 extends from the handle 112. The hook 114 includes a first end 128 distal from the handle 112 and a second end 130 connected to the handle 112 at or adjacent to the bend 122 of the handle 112. The hook 114 includes a curve 124 between the first and second ends 128, 130. In the representative embodiment of the invention, the second end 130 of the hook 114 may interfit with a connection port 132 disposed on an outer radius of the bend 122 of the handle 112. Preferably, the connection port 132 is oriented tangential to the bend 122. The connection port 132 includes an opening 134 formed therein and configured to receive an interfit element 136 of the hook 114 disposed at the second end 130 of the hook 114. As shown in FIG. 4, the interfit element 136 may extend perpendicularly from the second end 130 of the hook 114 in a first direction.

As a result, when the interfit element 136 of the hook 114 is disposed within the opening 134 of the connection port 132, the hook 114 extends outward from the bend 122 before gently curving at the curve 124. The curve 124 is configured to allow the hook 114 of the retractor 110 to be placed around a femur, such as femur 38 shown in FIG. 3. As a result, varying embodiments of the invention may include hook elements 114 having a variety of orientations and sizes with varying angles of extension from the bend 122 of the handle 112, varying radii of curvature of the cure 124 of the hook 114, and/or varying lengths and thicknesses. Further yet, the detachability of the hook 114 from the handle 112 allows for the hook 114 to be interchanged with varying hooks 114.

FIG. 4 further depicts a tool 115 that may be used to couple the hook 114 to the handle 112. As shown, the hook 114 may include a connection element 137 extending perpendicular from the second end 130 of the hook 114 in a second direction. The connection element 137 is configured to interfit with the tool 115 to allow a tool to hold the hook 114 while the interfit element 136 of the hook 114 is interfit with the connection port 132 of the handle 112 to couple the hook 114 to the handle 112. Preferably, the second direction being opposite of the first direction. However, in alternative embodiments of the invention the interfit element 136 and connection element 137 of the hook 114 may extend from the second end 130 of the hook 114 in any direction and at any angle.

Next, the acetabular component 116 of the retractor 110 may be detachably coupled to the second end 120 of the handle 112. As shown in FIG. 3, the acetabular component 116 is configured to sit within the acetabulum 44 during use of the retractor 1110. The acetabular component 116 includes a ball 146 that is sized to fit within the acetabulum 44. The ball 146 may be sized in variety of sizes, including, but not limited to, 32 mm, 36 mm, 40 mm, 44 mm, 46 mm, 50 mm, and other commonly used sizes in hip replacement surgery.

In this embodiment of the invention, an opening 142 may be formed in a first end 147 of the ball 146. An interfit element 140, such as a snap fit connection, may be disposed at the second end 120 of the handle 112. The interfit element 140 is configured to interfit within the opening 142 of the ball 146 to removably couple the ball 142 of the acetabular component 116 to the handle 112. In some instances, the first end 147 of the ball 146 may be a flat surface to assist in receiving the interfit element 140.

As stated above, the orthopedic retractor described herein is usable in a variety of hip replacement approaches. While the direct anterior approach is highlighted herein, this retractor can be used with any hip approach and any operating table, such as, but not limited to any anterior-based hip approach, direct and other lateral-based hip approaches, Watson-Jones hip approach, Rottinger hip approach, anterior based muscle sparing, and posterior hip approaches.

It should be understood that the above description, while indicating representative embodiments of the present invention, is given by way of illustration and not of limitation. Many changes and modifications may be made within the scope of the present invention without departing from the spirit thereof, and the invention includes all such modifications. Various additions, modifications, and rearrangements are contemplated as being within the scope of the following claims, which particularly point out and distinctly claim the subject matter regarding as the invention, and it is intended that the following claims cover all such additions, modifications, and rearrangements.

What is claimed is:

1. An orthopedic retractor comprising:
a handle having a first end and a second end;
an acetabular component removably coupled to the second end of the handle;
a hook element having a first end removably coupled to the handle at a location between the first and second ends, a second end, and a curve disposed between the first and second ends.

2. The orthopedic retractor of claim 1 wherein the handle further includes a bend disposed between the first and second ends; and
wherein the hook element is removably coupled to the handle at the bend of the handle.

3. The orthopedic retractor of claim 2 wherein the handle includes a connection port disposed on an outer radius of the bend; and
wherein the hook element includes an interfit element disposed at the first end of the hook element and configured to be disposed within the connection port to removably couple the hook element to the handle.

4. The orthopedic retractor of claim 3 wherein the connection port is oriented tangential to the outer radius of the bend.

5. The orthopedic retractor system of claim 3 wherein the interfit element extends from the first end of the hook element at an angle.

6. The orthopedic retractor system of claim 3 wherein hook element further includes a connection element extending from the first end of the hook and configured to interfit with a tool.

7. The orthopedic retractor system of claim 6 wherein the interfit element extends from the first end of the hook element in a first direction; and
wherein the connection element extends from the first end of the hook element in a second direction.

8. The orthopedic retractor system of claim 1 wherein the acetabular component includes a ball having an opening formed in a first end of the ball; and
wherein the second end of the handle includes an interfit element configured to interfit with the opening of the ball.

9. The orthopedic retractor system of claim 1 wherein the acetabular component includes a ball having a connection element extending outward therefrom; and
wherein the connection element is configured to receive the second end of the handle.

10. A method of using an orthopedic retractor comprising:
coupling an acetabular component to a second end of a handle;
disposing the acetabular component within an acetabulum;
placing a hook element around a femur so that the femur is partially surrounded by a curve of the hook element;
coupling a first end of the hook element to the handle; and
using the handle to pivot the retractor about the acetabulum to move the femur.

11. The method of claim 10 wherein coupling the first end of the hook element to the handle comprises coupling the first end of the hook element to an outer radius of a bend of the handle disposed between the first and second ends of the handle.

12. The method of claim 11 wherein coupling the first end of the hook element to an outer radius of a bend of the handle disposed between the first and second ends of the handle includes disposing an interfit element at the first end of the hook element within a connection port disposed on an outer radius of the bend.

13. The method of claim 10 wherein coupling the acetabular component to a second end of a handle includes disposing an interfit element at the second end of the handle within an opening formed in a first end of a ball of the acetabular component.

14. The method of claim 10 wherein coupling the acetabular component to a second end of a handle includes disposing a second end of the handle within a connection element extending outward from a ball of the acetabular component.

15. An orthopedic retractor comprising:
a handle having a first end, a second end, and a bend disposed between the first end and the second end;
a hook element removably coupled to the handle at or adjacent to the bend of the handle, the hook element extending from a first end at the handle to a second end spaced apart from the handle and having a curve disposed between the first and second ends, the curve configured to receive a femur of a patient; and
an acetabular component removably coupled to the second end of the handle, the acetabular component including a ball sized to fit within an acetabulum of the patient.

16. The orthopedic retractor of claim 15 wherein the handle further includes a connection port disposed on an outer radius of the bend; and
wherein the hook element further includes an interfit element disposed at the first end of the hook element, the interfit element configured to interfit with the connection port to removably couple the hook element to the handle.

17. The orthopedic retractor of claim 17 wherein the connection port is oriented tangential to the outer radius of the bend.

18. The orthopedic retractor of claim 17 wherein the interfit element extend from the first end of the hook element in a first direction.

19. The orthopedic retractor of claim 15 wherein the ball of the acetabular component includes an opening formed in a first end of the ball; and
wherein the second end of the handle includes an interfit element configured to interfit with the opening of the ball.

20. The orthopedic retractor system of claim 15 wherein the ball of the acetabular component includes a connection element extending outward therefrom, the connection element configured to receive the second end of the handle.
